(19) United States
(12) Patent Application Publication
(10) Pub. No.: US 2026/0097084 A1
(43) Pub. Date: Apr. 9, 2026

(54) PHARMACEUTICAL COMPOSITION CONTAINING CBFbeta-EXPRESSING VASCULAR SMOOTH MUSCLE CELLS OR CULTURE FLUID THEREOF AS ACTIVE INGREDIENT FOR PREVENTION OR TREATMENT OF AGE-RELATED DISEASES

(71) Applicant: KYUNGPOOK NATIONAL UNIVERSITY INDUSTRY-ACADEMIC COOPERATION FOUNDATION, Daegu (KR)

(72) Inventors: Je-Yong CHOI, Daegu (KR); Xiangguo CHE, Daegu (KR); Xian JIN, Daegu (KR)

(73) Assignee: KYUNGPOOK NATIONAL UNIVERSITY INDUSTRY-ACADEMIC COOPERATION FOUNDATION, Daegu (KR)

(21) Appl. No.: 19/114,149

(22) PCT Filed: Sep. 18, 2023
(86) PCT No.: PCT/KR2023/014083

§ 371 Date: Mar. 21, 2025

(30) Foreign Application Priority Data
Sep. 21, 2022 (KR) 10-2022-0119428
Sep. 14, 2023 (KR) 10-2023-0122311

(51) Int. Cl. A61K 35/44; A61K 38/17; A61K 45/00; A61P 9/00; A61P 19/10

(52) U.S. Cl. CPC A61K 35/44; A61K 38/1709; A61K 45/00; A61P 9/00; A61P 19/10

(57) ABSTRACT

The present invention relates to a pharmaceutical composition containing Cbfβ (Core-binding factor subunit beta) expressing vascular smooth muscle cells or a culture fluid thereof as an active ingredient for the prevention or treatment of age-related diseases. It has been found that inhibition of Cbfβ expression in vascular smooth muscle cells promotes vascular calcification and osteoclast differentiation and that the culture fluid of Cbfβ-expressing vascular smooth muscle cells inhibits osteoclast differentiation. Thus, the composition can be advantageously utilized as a pharmaceutical composition for the prevention, treatment, alleviation, or diagnosis of age-related diseases.

### TECHNICAL FIELD

The present disclosure relates to a pharmaceutical composition for preventing or treating a geriatric disease, including a core-binding factor subunit beta (Cbfβ)-expressing vascular smooth muscle cell or a culture fluid thereof as an active ingredient.

### BACKGROUND ART

The number of cases where cardiovascular and bone diseases occur simultaneously is rapidly increasing due to growth of the elderly population. Osteoporosis is a representative geriatric disease resulting from dysfunction of skeletal cells due to aging, vascular aging, and various inflammatory changes. Vascular calcification is a type of cardiovascular disease that can be caused by diabetes, kidney diseases, and osteoporosis. As bone mass decreases, the likelihood of developing vascular calcification increases, while there is a proportional relationship between chronic vascular diseases and osteoporosis. Biomolecules and proteins secreted from vascular cells may affect the activity of skeletal cells, and particularly, they promote the activity of osteoclasts among vascular cell-derived proteins, which results in destruction of bone tissues to cause osteoporosis. Accordingly, the development of new drugs that can treat cardiovascular diseases and bone diseases simultaneously is actively underway.

### DISCLOSURE OF THE INVENTION

### Technical Goals

An object of the present disclosure is to provide a pharmaceutical composition for preventing or treating a geriatric disease, including a core-binding factor subunit beta (Cbfβ)-expressing vascular smooth muscle cell or a culture fluid thereof as an active ingredient.

Another object of the present disclosure is to provide a health functional food composition for preventing or ameliorating a geriatric disease, including the cell or culture fluid thereof as an active ingredient.

Another object of the present disclosure is to provide a pharmaceutical composition for preventing or treating a geriatric disease, including an agent that induces expression or activity of a core-binding factor subunit beta (Cbfβ) protein or a gene encoding the same as an active ingredient.

Another object of the present disclosure is to provide a biomarker composition for diagnosing a geriatric disease, including a core-binding factor subunit beta (Cbfβ) protein or a gene encoding the same as an active ingredient.

Another object of the present disclosure is to provide a composition for diagnosing a geriatric disease, including an agent capable of identifying an expression level of the protein or a gene encoding the same as an active ingredient.

Another object of the present disclosure is to provide a kit for diagnosing a geriatric disease, including the composition for diagnosing a geriatric disease as an active ingredient.

Another object of the present disclosure is to provide a method of providing information for diagnosing a geriatric disease, including isolating a biological sample from an individual (first step); and measuring an expression level of a core-binding factor subunit beta (Cbfβ) protein or a gene encoding the same in the biological sample isolated in the first step (second step).

Another object of the present disclosure is to provide a method of screening a therapeutic agent for a geriatric disease, including treating a geriatric disease cell line with test substances (first step); measuring expression of a core-binding factor subunit beta (Cbfβ) protein or a gene encoding the same in the cell line of the first step (second step); and selecting a test substance which induces expression of the Cbfβ by comparing with a geriatric disease cell line which is not treated with the test substance (third step).

### Technical Solutions

To achieve the above objects, the present disclosure provides a pharmaceutical composition for preventing or treating a geriatric disease, including a core-binding factor subunit beta (Cbfβ)-expressing vascular smooth muscle cell or a culture fluid thereof as an active ingredient.

In addition, the present disclosure provides a health functional food composition for preventing or ameliorating a geriatric disease, including the cell or a culture fluid thereof as an active ingredient.

In addition, the present disclosure provides a pharmaceutical composition for preventing or treating a geriatric disease, including an agent that induces expression or activity of a core-binding factor subunit beta (Cbfβ) protein or a gene encoding the same as an active ingredient.

In addition, the present disclosure provides a biomarker composition for diagnosing a geriatric disease, including a core-binding factor subunit beta (Cbfβ) protein or a gene encoding the same as an active ingredient.

In addition, the present disclosure provides a composition for diagnosing a geriatric disease, including an agent capable of identifying an expression level of the protein or a gene encoding the same as an active ingredient.

In addition, the present disclosure provides a kit for diagnosing a geriatric disease, including the composition for diagnosing a geriatric disease as an active ingredient.

In addition, the present disclosure provides a method of providing information for diagnosing a geriatric disease, including isolating a biological sample from an individual (first step); and measuring an expression level of a core-binding factor subunit beta (Cbfβ) protein or a gene encoding the same in the biological sample isolated in the first step (second step).

In addition, the present disclosure provides a method of screening a therapeutic agent for a geriatric disease, including treating a geriatric disease cell line with test substances (first step); measuring expression of a core-binding factor subunit beta (Cbfβ) protein or a gene encoding the same in the cell line of the first step (second step); and selecting a test substance which induces expression of the Cbfβ by comparing with a geriatric disease cell line which is not treated with the test substance (third step).

### Advantageous Effects

According to the present disclosure, it has been found that, when expression of core-binding factor subunit beta (Cbfβ) is suppressed in vascular smooth muscle cells, vascular calcification and osteoclast differentiation are promoted, and a culture fluid of Cbfβ-expressing vascular smooth muscle cells suppresses osteoclast differentiation, such that the present disclosure may be useful as a composition for preventing, treating, ameliorating, or diagnosing a geriatric disease.

### BRIEF DESCRIPTION OF DRAWINGS

FIG. 1 shows results of analyzing an effect of Cbfβ on vascular homeostasis.

FIG. 2 shows results of analyzing an effect of Cbfβ on blood pressure. * p<0.05

FIG. 3 shows results of analyzing an effect of Cbfβ on vascular calcification.

FIG. 4 shows results of analyzing an effect of Cbfβ on bone metabolism.

FIG. 5 shows results of analyzing an effect of vascular smooth muscle cell-derived proteins on an activity of osteoclasts.

FIG. 6 shows results of analyzing an effect of a culture fluid of Cbfβ-deleted vascular smooth muscle cells on an activity and differentiation of osteoclasts.

FIG. 7 shows results of analyzing an effect of a culture fluid of Cbfβ-overexpressing vascular smooth muscle cells on osteoclast differentiation.

FIG. 8 shows results of analyzing an effect of Cbfβ on expression of genes associated with osteoclast differentiation.

### BEST MODE FOR CARRYING OUT THE INVENTION

Hereinafter, the present disclosure will be described in detail.

The present disclosure provides a pharmaceutical composition for preventing or treating a geriatric disease, including a core-binding factor subunit beta (Cbfβ)-expressing vascular smooth muscle cell or a culture fluid thereof as an active ingredient.

The Cbfβ-expressing vascular smooth muscle cell or culture fluid thereof may suppress blood pressure increase; or osteoclast differentiation.

The geriatric disease may be a vascular aging disease or a bone disease.

The vascular aging disease may be one or more selected from the group consisting of vascular calcification, osteoporosis, osteoarthritis, arteriosclerosis, fatty liver, liver fibrosis, diabetes, and dementia, but is not limited thereto.

The bone disease may be one or more selected from the group consisting of fracture, osteoarthritis, rheumatoid arthritis, osteoporosis, and osteomalacia, but is not limited thereto.

The pharmaceutical composition of the present disclosure may be prepared in the form of a unit dose by formulation using a pharmaceutically acceptable carrier or prepared by encapsulating into a multi-capacity container, in accordance with a method that may be easily carried out by a person with ordinary skill in the art to which the present disclosure pertains.

The pharmaceutically acceptable carriers are those commonly used in preparation, including, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may further include lubricants, humectants, sweeteners, flavoring agents, emulsifiers, suspensions, and preservatives, in addition to the above ingredients.

In the present disclosure, the content of the additives included in the pharmaceutical composition is not particularly limited and may be appropriately adjusted within a content range used in the conventional preparation.

The pharmaceutical composition may be formulated in the form of one or more external skin agents selected from the group consisting of, but are not limited to, injectable formulations such as aqueous solutions, suspensions, and emulsions, pills, capsules, granules, tablets, creams, gels, patches, sprays, ointments, plasters, lotions, liniments, pastas, and cataplasmas.

The pharmaceutical composition of the present disclosure may include a pharmaceutically acceptable carrier and diluent that are additionally present for formulation. The pharmaceutically acceptable carriers and diluents include, but are not limited to, excipients such as starch, sugars, and mannitol, fillers and extenders such as calcium phosphate, cellulose derivatives such as carboxymethylcellulose and hydroxypropylcellulose, binders such as gelatin, alginate, and polyvinyl pyrrolidone, lubricants such as talc, calcium stearate, hydrogenated castor oil, and polyethylene glycol, disintegrating agents such as povidone and crospovidone, and surfactants such as polysorbate, cetyl alcohol, and glycerol. The pharmaceutically acceptable carriers and diluents may be biologically and physiologically friendly to a subject. Examples of diluents may include, but are not limited to, brine, water-soluble buffers, solvents, and/or dispersion media.

The pharmaceutical composition of the present disclosure may be administered orally or parenterally (e.g., applied intravenously, subcutaneously, intraperitoneally, or topically) depending on the desired method. When administered orally, it may be formulated as tablets, troches, lozenges, water-soluble suspensions, oil-based suspensions, powder preparation, granules, emulsions, hard capsules, soft capsules, syrups, and elixirs. When administered parenterally, it may be formulated as injection solutions, suppositories, powders for respiratory inhalation, aerosols for sprays, ointments, powders for application, oils, and creams.

The dosage of the pharmaceutical composition of the present disclosure may vary depending on the patient's condition, weight, age, sex, health status, dietary constitution specificity, nature of the preparation, severity of disease, administration time for composition, method of administration, duration or interval of administration, excretion rate, and drug form, and may be appropriately selected by a person skilled in the art. For example, it may range from about 0.1 to 10,000 mg/kg but is not limited thereby, and it may be administered once or several times a day.

The pharmaceutical composition may be administered orally or parenterally (e.g., applied intravenously, subcutaneously, intraperitoneally, or topically) depending on the desired method. The pharmaceutically effective amount and effective dosage of the pharmaceutical composition of the present disclosure may vary by the preparation method of the pharmaceutical composition, type of administration, administration time, and administration route, and a person with ordinary skill in the art may easily determine and prescribe the effective dosage for the desired treatment. The administration of the pharmaceutical composition of the present disclosure may be conducted once a day or in several divided doses.

In addition, the present disclosure provides a health functional food composition for preventing or ameliorating a geriatric disease, including core-binding factor subunit beta (Cbfβ)-expressing vascular smooth muscle cells or a culture fluid thereof as an active ingredient.

The present disclosure may be used generally as a food product that is conventionally used.

The food composition of the present disclosure may be used as a health functional food. The term “health functional food” as used herein refers to a food manufactured and processed using raw materials or ingredients that have useful functionality for the human body in accordance with the Health Functional Food Act, and the term “functionality” as used herein refers to consumption for the purpose of deriving useful effects for health purposes such as adjusting nutrients or physiological actions on the structure and function of the human body.

The health functional food composition may include conventional food additives, and the suitability as the “food additive” is determined by the standards and criteria associated with corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills. For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling the same in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited and includes all health functional foods in the ordinary sense.

The term “prevention” as used herein refers to any action of suppressing or delaying geriatric diseases by administering the composition according to the present disclosure.

The term “treatment” as used herein refers to any action of ameliorating or favorably changing the geriatric diseases by administering the composition according to the present disclosure.

The term “amelioration” as used herein refers to any action of making the bad condition of geriatric diseases better by administering the composition according to the present disclosure.

In addition, the present disclosure provides a pharmaceutical composition for preventing or treating a geriatric disease, including an agent that induces expression or activity of a core-binding factor subunit beta (Cbfβ) protein or a gene encoding the same as an active ingredient.

In addition, the present disclosure provides a biomarker composition for diagnosing a geriatric disease, including a core-binding factor subunit beta (Cbfβ) protein or a gene encoding the same as an active ingredient.

The geriatric disease may be a vascular aging disease or a bone disease.

The protein or gene encoding the same may have decreased expression in vascular smooth muscle cells of patients with a geriatric disease.

As used herein, the term “diagnosis” refers to identification of the presence or characteristics of a pathological condition, and for the purposes of the present disclosure, it refers to identification of a geriatric disease, including determining a subject's susceptibility to a geriatric disease or at least one or more symptoms thereof as well as therametrics (e.g., monitoring the condition of a subject to provide information on the therapeutic efficacy). It also includes the primary diagnosis of a clinical state or the diagnosis of a relapsed disease.

As user herein, the term “biomarker” refers to an indicator that may detect changes in the body as a substance capable of identifying the normal or pathological state of a living organism and changes thereof, may include organic biomolecules such as polypeptides, nucleic acids, lipids, glycolipids, glycoproteins, and sugars (monosaccharides, disaccharides, and oligosaccharides), and may be used to diagnose a geriatric disease as in the present disclosure.

In addition, the present disclosure provides a composition for diagnosing a geriatric disease, including an agent capable of identifying an expression level of a core-binding factor subunit beta (Cbfβ) protein or a gene encoding the same as an active ingredient.

The agent capable of identifying the expression level may be an antibody, peptide, aptamer, or compound that specifically binds to the protein; or a primer or probe that specifically binds to a gene encoding the protein, but is not limited thereto.

As used herein, the term “primer” refers to a short nucleic acid sequence with a short free 3′ hydroxyl group, which may form base pairs with a complementary template and serves as a starting point for replication of template strands. A primer may initiate DNA synthesis in the presence of reagents for polymerization (i.e., DNA polymerase or reverse transcriptase) and four different nucleotide triphosphates in an appropriate buffer and temperature. PCR conditions and lengths of sense and antisense primers may be appropriately selected according to techniques known in the art.

As used herein, the term “probe” refers to a fragment of several to several hundred base sequences capable of specifically binding to a gene or miRNA and enables identification of an expression level of the gene since it is labeled. Appropriate probes and hybridization conditions may be appropriately selected according to techniques known in the art.

In addition, the present disclosure provides a kit for diagnosing a geriatric disease, including the composition for diagnosing a geriatric disease as an active ingredient.

The kit may be used to diagnose the onset of a geriatric disease by measuring the expression level of the protein or gene encoding the same in a sample isolated from an individual suspected of having a geriatric disease.

Additionally, the kit may include an agent for measuring the expression level of the protein or gene encoding the same as well as one or more other types of component compositions, solutions, or devices suitable for the analytical method.

For example, a kit according to the present disclosure may be one that includes genomic DNA derived from a sample to be analyzed for performing PCR, a primer set specific for a marker gene of the present disclosure, an appropriate amount of DNA polymerase, a dNTP mixture, a PCR buffer solution, and water. The PCR buffer solution may include KCl, Tris-HCl, and MgCl₂. In addition, components necessary for performing electrophoresis to identify whether a PCR product has been amplified may be additionally included in the kit of the present disclosure.

Additionally, the kit according to the present disclosure may be one that includes essential elements required for performing RT-PCR. In addition to each primer pair specific for a marker gene, the RT-PCR kit may include a test tube or other appropriate containers, reaction buffers, deoxynucleotides (dNTPs), enzymes such as Taq polymerase and reverse transcriptase, DNase and RNase inhibitors, DEPC water, and sterile water. In addition, it may include primer pairs specific for the genes used as quantitative controls.

In addition, the present disclosure provides a method of providing information for diagnosing a geriatric disease, including isolating a biological sample from an individual (first step); and measuring an expression level of a core-binding factor subunit beta (Cbfβ) protein or a gene encoding the same in the biological sample isolated in the first step (second step).

The biological sample may be a vascular smooth muscle cell.

A method of measuring the protein expression level may be one or more selected from, but is not limited to, the group consisting of Western blot, enzyme linked immunosorbent assay (ELISA), radioimmunoassay (RIA), radioimmunodiffusion, Ouchterlony immunodiffusion, rocket immunoelectrophoresis, histoimmunostaining, immunoprecipitation assay, complement fixation assay, FACS, and protein chip.

A method of measuring the gene expression level may be one or more selected from the group consisting of, but is not limited to, microarray, next generation sequencing (NGS), polymerase chain reaction (PCR), reverse transcription PCR (RT-PCR), competitive RT-PCR, real-time RT-PCR, RNase protection assay (RPA), Northern blotting, and DNA chip.

In addition, the present disclosure provides a method of screening a therapeutic agent for a geriatric disease, including treating a geriatric disease cell line with test substances (first step); measuring expression of a core-binding factor subunit beta (Cbfβ) protein or a gene encoding the same in the cell line of the first step (second step); and selecting a test substance which induces expression of the Cbfβ by comparing with a geriatric disease cell line which is not treated with the test substance (third step).

As used herein, the term “test substance” refers to an unknown candidate substance used in screening to examine whether it affects the expression level of a gene or affects the expression or activity of a protein. The test substance includes, but is not limited to, chemicals, nucleic acids, antisense RNA, small interference RNA (siRNA), and natural extracts.

### MODES FOR CARRYING OUT THE INVENTION

Hereinafter, the present disclosure will be described in more detail through examples to help understanding of the present disclosure. However, examples below are merely intended to illustrate the present disclosure, and the scope of the present disclosure is not limited to the following examples. Examples of the present disclosure are provided to more completely explain the present disclosure to those skilled in the art.

### [Experimental Example 1] Preparation for Experiments

### 1-1. Reagent

The reagents (materials) used in the experiment and the suppliers are as shown in Table 1 below.

TABLE 1

| Reagent | Supplier |
|---|---|
| DMSO (dimethyl sulfoxide) | Sigma |
| ethanol | |
| fast red violet LB | |
| glycerol | |
| napthol As-Mx phosphate | |
| N,N-dimethylformamide | |
| nitric acid | |
| nonylphenyl-polyethylene- | |
| glycol acetate | |
| PFA (paraformaldehyde) | |
| acetone | JUNSEI |
| formaldehyde solution | (Nihonbashi-honcho, |
| sodium acetate trihydrate | Chuo-ku, Tokyo) |
| sodium tartrate dihydrate | |
| α-MEM (α-minimum essential | Thermo scientific |
| medium) hyclone | (Rockford, IL, U.S.A) |
| permount | |
| FBS (Fetal bovine serum) | GIBCO |
| | (Grand Island, NY, U.S.A) |
| M-CSF (human macrophage-colony | PEPROTECH, INC. (Rocky Hill, |
| stimulation factor) | NJ, USA) |
| N,N-dimethyl-p-toluidine | MP Biomedicals (Solon-Ohio, |
| | BC, U.S.A.) |
| Penicillin/Streptomycin | Lonza (Rockland, ME, USA) |
| Red blood cell lysate | BioLegend (San Diego, |
| | CA, U.S.A.) |
| RANKL (receptor activator for | R&D systems (Minneapolis, |
| nuclear factor κB ligand) | MN, U.S.A.) |

### 1-2. Laboratory Animals

Cbfβ^{f/f} 16-week-old male mice were provided by RIKEN, Japan, Sm22a Cre 16-week-old male mice (Sm22a Cre-overexpressing mice) by the laboratory of Professor Cho Je-yeol at Seoul National University, and ROR26 mice by the laboratory of Professor Kim Jeong-eun at Kyungpook National University. When mating Sm22a Cre mice with Cbfβ^{f/f} mice, on the basis of the principle that the Cbfβ gene is specifically deleted in the vascular smooth muscle cells of the mated mice (Sm22a Cre;Cbfβ^{f/f}), Cbfβ-deleted mice were created. In addition, when mating Sm22a-Cre mice with ROR26 mice, it is possible to observe the loci where Sm22a-Cre is expressed in the mated mice through X-GAL staining, so mating was carried out. Specifically, the mice used in the experiment are as follows.
1) Cbfβf/f mice: normal mice (control group; WT)
2) Sm22a Cre;Cbfβ^{f/f} mice (Cbfβ^{Δvc/+}): heterozygous Cbfβ-deleted mice (HE)
3) Sm22a Cre;Cbfβ^{f/f} mice (Cbfβ^{Δvc/Δvc}): homozygous Cbfβ-deleted mice (HO)
4) Sm22a-Cre;ROR26 mice: Sm22a Cre-overexpressing mice

The experiment was conducted after acclimatization for one week in a specific pathogen free (SPF) laboratory animal breeding room (at 25° C. with relative humidity of 60%). The animals were fed a regular diet (Super bead Co, Korea), and all animal experiments were conducted in accordance with the regulations of the Institutional Animal Care and Use Committee of Kyungpook National University (Institutional Animal Approval Number: KNU 2021-0099).

### 1-3. Cell

Mouse bone marrow monocytes (mBMM) were isolated from the femurs and tibias of 8-week-old mice. The femur and tibia were separated from mice that had cervical dislocation to remove the muscles, both ends were cut, and then bone marrow cells were isolated by passing α-MEM using a syringe. The separated bone marrow cells were centrifuged, red blood cell lysis solution was added to hemolyze the red blood cells, and then medium [α-MEM, 10% (v/v) FBS, penicillin (100 units/ml), and streptomycin (100 units/ml)] was added, followed by culture in a constant temperature and humidity incubator at 37° C. in the presence of 5% (v/v) CO₂ for 1 day. Only cells that were floating without being attached to the culture dish were collected, treated with M-CSF (20 ng/ml), and cultured for 3 days to obtain bone marrow-derived monocytes/macrophages. To differentiate macrophages into osteoclasts, M-CSF (30 ng/ml) and RANKL (30 ng/ml) were added and cultured for 4 days. During differentiation induction, the medium was changed every two days.

### [Experimental Example 2] Real-Time PCR

To identify the changes in gene expression of osteoclast differentiation markers, real-time PCR was performed. Total RNA was extracted from differentiated osteoclasts using an RNA extraction kit (Easy-blue), and 2 μg of total RNA was synthesized into cDNA using reverse transcriptase. Real-time PCR was performed by mixing 0.5 μl of the synthesized cDNA with 2×SYBR green PCR master mixture (5 μl) and specific primer (0.2 μl). The primers used for real-time PCR are as listed in Table 2 below, and the primers were designed 10 using Primer Express software (ABI).

TABLE 2

| | | |
|---|---|---|
| mDCSTAMP | Forward | CTTCCGTGGGCCAGAAGTT |
| | Reverse | AGGCCAGTGCTGACTAGGATGA |
| mc-fms | Forward | CCTGAATCTCCCGGAAGCA |
| | Reverse | CAAGCTCGGTACAACGGTAGGT |
| mRank | Forward | TCTGCAGCTCTTCCATGACACT |
| | Reverse | GAAGAGGAGCAGAACGATGAGACT |
| mTRAP | Forward | TCCCCAATGCCCCATTC |
| | Reverse | CGGTTCTGGCGATCTCTTTG |
| mGapdh | Forward | TGTGTCCGTCGTGGATCTGA |
| | Reverse | CCTGCTTCACCACCTTCTTGA |
| mc-fos | Forward | GCATGGGCTCTCCTGTCAA |
| | Reverse | GGCACTAGAGACGGACAGATCTG |
| mOscar | Forward | ACCTGGCACCTACTGTTGCTATTAC |
| | Reverse | GCTGGCTGCGCTGTGAT |
| mCathepsin | Forward | GGCTGTGGAGGCGGCTAT |
| K | Reverse | AGAGTCAATGCCTCCGTTCTG |
| mVegf | Forward | CAGGCTGCTGTAACGATGAA |
| | Reverse | GCATTCACATCTGCTGTGCT |
| mRankL | Forward | GATTTTTCAAGCTCCGAGCTGG |
| | Reverse | CCTGAACTTTGAAAGCCCCAA |
| mOpg | Forward | ACTCGAACCTCACCACAGAGCA |
| | Reverse | CGCTCGATTTGCAGGTCTTTC |
| mRunx1 | Forward | CAGGCAGATCCAGCCATC |
| | Reverse | TTGAGAGTCGACTGGAAAGTTCT |
| mRunx2 | Forward | GCACAAACATGGCCAGATTCA |
| | Reverse | AAGCCATGGTGCCCGTTAG |
| mCbfμ | Forward | GTTTACAGCTCTTTGGGTTCCA |
| | Reverse | TTACTGCCAGCAGCTGTGA |
| mSmmhc | Forward | GCGCAATACCACGCCTAACTT |
| | Reverse | AGATGCGGATGCCTTCCAA |
| mPPARG | Forward | GAAAGACAACGGACAAATCACC |
| | Reverse | GGGGGTGATATGTTTGAACTTG |
| mPDGFB | Forward | CAAGAGTGTGGGCAGGGTTA |
| | Reverse | CATCGAGACAGACGGACGAG |
| mPDGFRB | Forward | AAGCTGCAGGTCAATGTCCC |
| | Reverse | CTCTGCAGGTAGACCAGGTG |
| mElastin | Forward | GGGATAAAACGAGGCGCTGA |
| | Reverse | AGCTCCTGGAACCCCTCC |
| mAngpt14 | Forward | GTTTGCAGACTCAGCTCAAGG |
| | Reverse | CCAAGAGGTCTATCTGGCTCTG |

### [Experimental Example 3] Tartrate Resistant Acid Phosphatase (Trap) Staining

Bone marrow mononuclear cells isolated from the femur and tibia of mice were treated with M-CSF (20 ng/ml) and reacted for 1 day, and then the cells attached to the culture dish were collected and seeded at 3×10⁴ cells/well in a 48-well multi plate, followed by culture for 4 days while replacing with osteoclast differentiation inducing medium. After removing the medium and washing with phosphate-buffered saline (PBS), the cells fixed with 10% formaldehyde solution were treated with Trap staining solution [Naphthol As-Mx, Fast red violet LB, and N,N-dimethylformamide dissolved in 50 mM acetate buffer (pH 5.0)] and stained at 37° C. for 30 minutes. The solution was removed, washed twice with PBS, and then observed under a microscope.

### [Experimental Example 4] Hematoxylin & Eosin (H&E) Staining

To observe the morphology of blood vessels, H&E staining was performed. After rehydration of the paraffin sections of blood vessels, the cell nuclei were stained with hematoxylin, and the cytoplasm was stained with eosin, followed by observation under a microscope.

### [Experimental Example 5] Verhoeff-Van Gieson (VVG) Staining

To observe the elastic fibers of blood vessels, VVG staining was performed. After rehydration of the paraffin sections of blood vessels, the sections were reacted in Verhoeff's solution for 1 hour, washed three times in running water, and reacted with 2% ferric chloride for 2 minutes. After washing in running water, the sections were reacted with 5% sodium thiosulfate for 5 minutes, washed again, stained with Van Gieson's solution, and observed under a microscope. All procedures were proceeded according to the method disclosed on the following site (https://www.ihcworld.com/_protocols/special_stains/vvg.htm).

### [Experimental Example 6] Tissue Immunohistochemistry Staining

After rehydration of the paraffin sections of the blood vessel, sections were placed in Tri-EGTA (Tris 1.211 g+EGTA 0.19 g/ddH₂O 1 L; TEG) solution, boiled in a microwave, retrieved, and slowly cooled to room temperature. Retrieved samples were blocked with 3% H₂O₂/methanol and 1% BSA/PBS solution and permeabilized with 0.1% Triton X-100. Afterwards, the primary antibody was reacted overnight at 4° C., and the secondary antibody was reacted at room temperature for 1 hour. Finally, the color was developed using a DAB substrate, and dehydration and mounting were performed, followed by observation under a microscope.

### [Experimental Example 7] Von Kossa Staining

To observe the degree of bone mineralization, Von Kossa staining was performed. The vertebrae were fixed in 4% PFA at 4° C. for 1 day. Paraffin samples were prepared without decalcification after fixation. After rehydrating the paraffin sections, 1% silver nitrate (AgNO₃) was added and reacted under ultraviolet (UV) light for 5 minutes. After the reaction was completed, the mixture was washed twice with third solution, added with 5% sodium thiosulfate, and reacted for 5 minutes, followed by removal of nonspecific signals. Afterwards, mounting was followed, and observation was conducted under a microscope.

### [Experimental Example 8] Tissue Immunohistochemistry Staining

After rehydration, the paraffin sections of blood vessels were blocked with 1% bovine serum albumin (BSA) for 1 hour and reacted with CD31 antibody for 1 hour at room temperature. Afterwards, the section was reacted with a secondary antibody conjugated with horse radish peroxidase (HRP) for 1 hour, then developed with color using 3,3′-diaminobenzidine (DAB) (Dako, Carpinteria, CA, USA), mounted, and observed under a microscope.

### [Experimental Example 9] Fluorescence Immunohistochemistry Staining

After rehydration, the paraffin sections of blood vessels were blocked with 1% BSA for 1 hour and reacted with PCNA antibody for 1 hour at room temperature. Afterwards, the sections were reacted with a fluorescence-conjugated secondary antibody for 1 hour, and then the cell nuclei were stained with 4′,6-diamidino-2-phenylindole (DAPI), mounted, and observed under a microscope.

### [Experimental Example 10] X-Gal Staining

The whole tissue was fixed in 4% paraformaldehyde (PFA)/0.1 M calcium phosphate solution [pH 7.4, 2 mM MgCl₂, 5 mM ethylene glycol tetra-acetic acid (EGTA)] for 30 minutes. Afterwards, the tissue was washed three times and stained in X-gal staining solution overnight at 37° C. The stained samples were made into paraffin blocks, cut into sections, and counter-stained with Nuclear Fast Red (Sigma Aldrich, St. St. Louis, MO, USA).

### [Experimental Example 11] Micro-CT Analysis

The mouse femurs were separated to remove muscles, fixed in 4% PFA at 4° C. for one day, washed three times in PBS solution the next day, and stored in PBS. The stored femurs were scanned in 6 μm unit from 1.6 mm to 2.3 mm below the growth plate using Skysan 1272 (Bruker, Massachusetts, USA) model, followed by analysis. Using micro-CT, spongy bone volume (BV/TV), spongy bone thickness (Tb.Th), spongy bone marrow (Tb.N), spongy bone area (Tb.Sp), and spongy bone mineral density (BMD) were measured, and bone volume (BV/TV) and bone mineral density (BMD) of cortical bone were measured.

### [Experimental Example 12] Measurement of Bone Strength

After removing the muscles by separating the mouse femurs, they were placed in 0.9% sodium chloride (pH 7.4) and stored at −20° C. Before analyzing the sample, the muscle was slowly thawed at 4° C., and the max load (N) and slope (N/mm) were measured using Instron (Tensile Tester, Williamston, SC, USA).

### [Experimental Example 13] Analysis of Blood Biochemistry

To determine the degree of osteoclast absorption in vivo, the secretion of CTX in serum was measured using ELISA. All experimental procedures were proceeded according to the standard manual of cloud clone (WUHAN, PRC).

### [Experimental Example 14] Measurement of Blood Pressure

To determine the effect of Cbfβ deletion on blood pressure, mouse blood pressure was measured. Before measuring blood pressure of mice, they were allowed to stabilize for 5 minutes, followed by measurement using an automatic blood pressure monitor (Tango+, Suntech, NC, USA).

### [Experimental Example 15] Alizarin Red Staining

To determine the effect of Cbfβ deletion on vascular calcification in a model of vascular calcification induced with high-dose vitamin D, Alizarin Red staining was performed. Kidneys and blood vessels isolated from sacrificed mice were washed three times with PBS for 10 minutes each, fixed with 4% PFA, washed three times with third distilled water for 10 minutes each, and reacted with Alizarin Red solution for 10 minutes. After washing three times with third distilled water, observation was followed under a microscope.

### [Experimental Example 16] MTS Assay

To determine the effect on proliferation of osteoblasts and osteoclasts, MTS assay was performed. MC3T3-E1 cells were seeded at 3×10⁴ cells/well in a 48-well multi-plate, then 24 hours later, the culture fluid obtained from vascular smooth muscle cells was treated followed by culture for 24 hours, and 20 μl of MTS solution was added to each well along with 100 μl of culture fluid, followed by culture for 4 hours. After 4 hours, the absorbance was measured at a wavelength of 490 nm using an ELISA reader.

### [Example 1] Analysis of Vascular Homeostasis

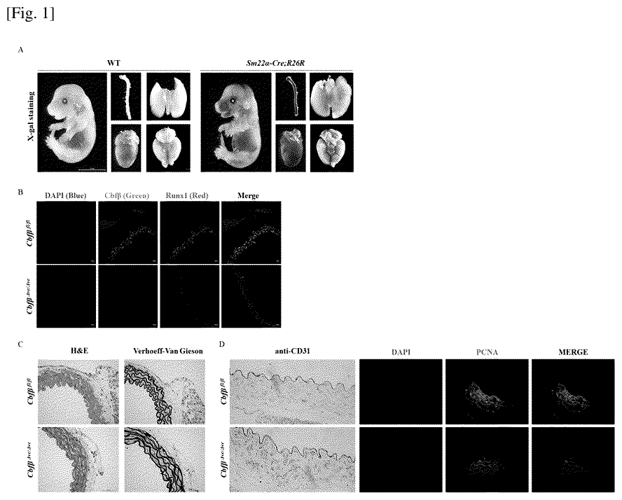

As a result of analyzing the effect of Cbfβ on vascular homeostasis in vascular smooth muscle cells, as shown in FIG. 1A, an increase in Sm22a-cre expression in vascular smooth muscle cells derived from Sm22a-Cre-overexpressing mice (Sm22a-Cre;ROR26) was observed through X-gal staining (Experimental Example 10). In addition, as shown in FIG. 1B, it was found through fluorescence immunohistochemistry staining (Experimental Example 9) that expression of Cbfβ and Runx1 was reduced in vascular smooth muscle cells derived from Cbfβ-deleted mice (Cbfβ^{Δvc/Δvc}). In addition, as shown in FIG. 1C, it was found through H&E and VVG staining (Experimental Examples 4-5) that the elastic fibers of the vascular smooth muscle derived from the Cbfβ-deleted mice were flat and irregular, and as shown in FIG. 1D, it was found through fluorescence immunohistochemistry staining (Experimental Example 9) that CD31 expression increased and vascular smooth muscle cell proliferation decreased in the vascular smooth muscle cells derived from the Cbfβ-deleted mouse. From the results, it was found that Cbfβ was involved in vascular homeostasis.

### [Example 2] Analysis of Blood Pressure

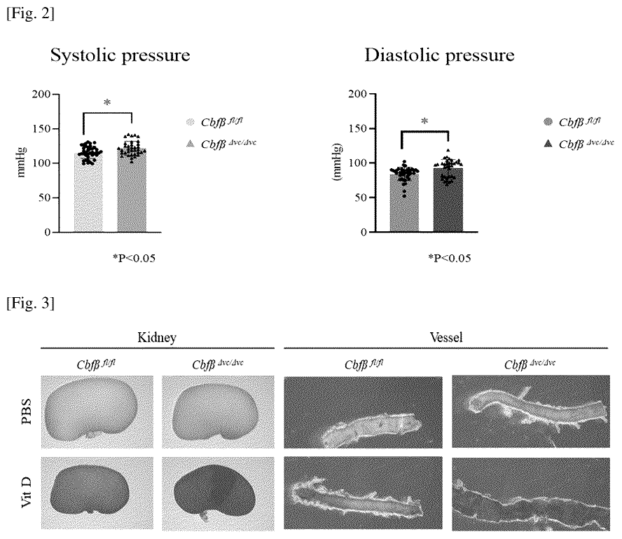

As a result of analyzing the effect of Cbfβ on blood pressure in vascular smooth muscle cells (Experimental Example 12), as shown in FIG. 2, it was found that blood pressure was higher in Cbfβ-deleted mice (Cbfβ^{Δvc/Δvc}) than in normal mice (Cbfβ^{fl/fl}) (n=4-5/group, *p<0.05). From the results, it was found that Cbfβ was involved in blood pressure homeostasis.

### [Example 3] Analysis of Vascular Calcification

To determine the effect of Cbfβ on vascular calcification in vascular smooth muscle cells, high-dose vitamin D (6×10⁵ IU/Kg) was subcutaneously injected into mice to induce vascular calcification, and vascular calcification was observed in the kidney and arteries via Alizarin Red staining (Experimental Example 13). When PBS was administered instead of vitamin D as a control, it was also administered by subcutaneous injection. Specifically, the experimental group was set up as follows.
1) PBS administration (200 μl) to normal mice (WT) (n=2)
2) Vitamin D administration (6×10⁵ IU/Kg) to normal mice (WT) (n=4)
3) PBS administration (200 μl) Cbfβ-deleted mice (Cbfβ^{Δvc/Δvc}) (n=2)
4) Vitamin D administration (6×10⁵ IU/Kg) to Cbfβ-deleted mice (Cbfβ^{Δvc/Δvc}) (n=4)

As a result, as shown in FIG. 3, vascular calcification was promoted in the kidneys and aorta of normal mice (Cbfβ^{fl/fl}) and Cbfβ-deleted mice (Cbfβ^{Δvc/Δvc}) administered with vitamin D, and vascular calcification was more significant in Cbfβ-deleted mice compared to normal mice. From the results, it was found that Cbfβ suppressed vascular calcification.

### [Example 4] Analysis on Bone Metabolism

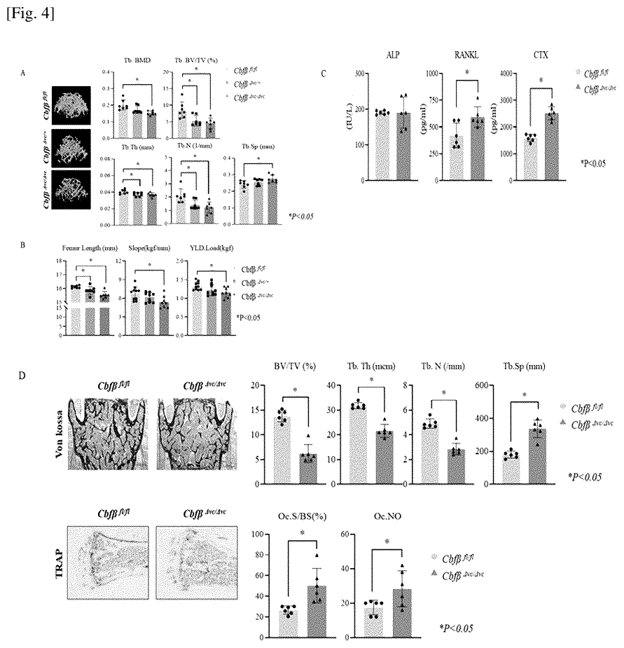

As a result of analyzing the effect of Cbfβ on bone metabolism in vascular smooth muscle cells, as shown in FIGS. 4A to 4B, it was observed through micro-CT and Instron (Experimental Examples 9-10) that the spongy bone density (Tb. BMD), spongy bone mass (Tb. BV/TV), spongy bone thickness (Tb.Th), and spongy bone marrow (Tb.N) of Cbfβ-deleted mice (Cbfβ^{Δvc/+} and Cbfβ^{Δvc/Δvc}) significantly decreased compared to normal mice (Cbfβ^{fl/fl}), spongy bone area (Tb.Sp) significantly increased, and bone strength indices such as femur length (Femur Length), slope, and YLD.load values decreased. In addition, as shown in FIG. 4C, it was found that CTX and RANKL expression increased in Cbfβ-deleted mice (Cbfβ^{Δvc/Δvc}) compared to normal mice through blood biochemical analysis (Experimental Example 11), as shown in FIG. 4D, the number of osteoclasts (Oc.NO) and osteoclast unit surface area/bone unit surface area (Oc. S/BS) increased in Cbfβ-deleted mice (Cbfβ^{Δvc/Δvc}) compared to the normal mice through TRAP staining (Experimental Example 3), bone volume (BV/TV), bone thickness (Tb.Th), and spongy bone number (Tb.N) decreased in the vertebrae, and the spongy bone area (Tb.Sp) increased through Von Kossa staining (Experimental Example 7). From the above results, it was found that Cbfβ was involved in bone metabolism homeostasis.

### [Example 5] Analysis of Activity and Differentiation of Osteoclasts

### 5-1. Vascular Smooth Muscle Cells

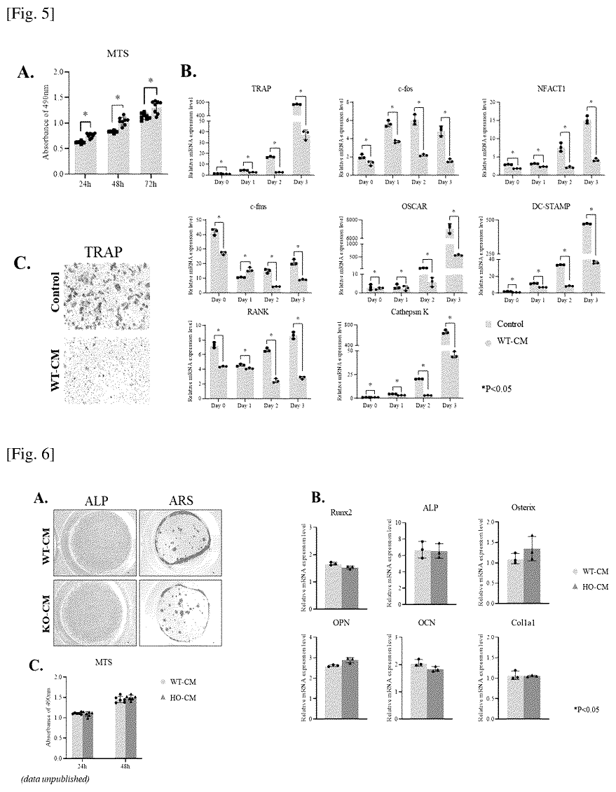

As a result of analyzing the effect of proteins derived from vascular smooth muscle cells on osteoclast activity and differentiation, as shown in FIGS. 5A and 5C, it was found that the vascular smooth muscle cell culture fluid-treated group (WT-CM, CM=vascular smooth muscle cell derived conditional media) promoted the proliferation of osteoclasts (monocytes) while suppressing the differentiation of osteoclasts compared to the control group (untreated group; Control) through MTS assay (Experimental Example 16) and Trap staining (Experimental Example 3). In addition, as shown in FIG. 5B, it was found through real-time PCR (Experimental Example 2) that the expression of osteoclast differentiation markers (c-fos, c-fms, RANK, TRAP, OSCAR, NFACT1, Cathepsin K, and DC-STAMP) was reduced in the vascular smooth muscle cell culture fluid-treated group compared to the control group. From the above results, it was determined that vascular smooth muscle cell-derived protein was involved in bone metabolism by promoting osteoclast proliferation and suppressing osteoclast differentiation.

### 5-2. Cbfβ-Deleted Vascular Smooth Muscle Cells

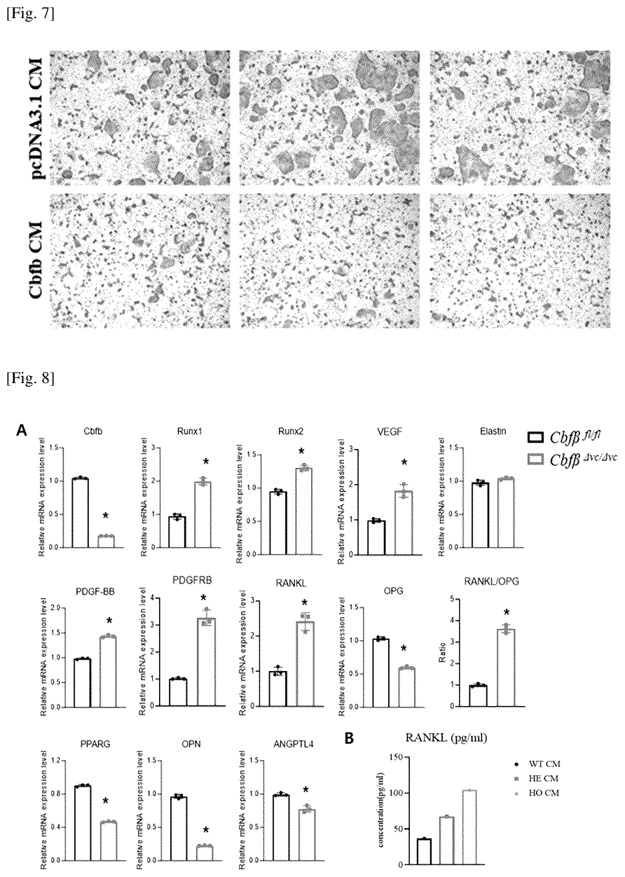

As a result of analyzing the effect of Cbfβ-deleted vascular smooth muscle cell culture fluid on osteoclast activity and differentiation, as shown in FIGS. 6A and 6C, it was found through MTS assay (Experimental Example 16) and Trap staining (Experimental Example 3) that, while osteoclast proliferation did not show a significant difference among the experimental groups, osteoclast differentiation increased in the Cbfβ-deleted vascular smooth muscle cell culture fluid-treated group (HO-CM) compared to the control group (WT-CM; normal vascular smooth muscle cell culture fluid-treated group, CM=vascular smooth muscle cell derived conditional media). In addition, as shown in FIG. 6B, it was determined through real-time PCR (Experimental Example 2) that RUNX2 and OCN expression decreased, Osterix and OPN expression increased, and ALP and Colla expression did not show significant differences between the experimental groups in the Cbfβ-deleted vascular smooth muscle cell culture fluid-treated group compared to the control. In addition, as shown in FIG. 7, it was found through Trap staining (Experimental Example 3) that osteoclast differentiation was reduced in the Cbfβ-overexpressing vascular smooth muscle cell culture fluid-treated group (Cbfβ CM) compared to the control group (normal vascular smooth muscle cell culture fluid-treated group; pcDN3.1 CM). From the results, it was found that Cbfβ suppressed osteoclast differentiation.

### [Example 6] Analysis on Expression of Genes Associated with Osteoclast Differentiation

As a result of analyzing the effect of Cbfβ on the expression of genes associated with osteoclast differentiation in vascular smooth muscle cells, as shown in FIG. 8A, it was found through real-time PCR (Experimental Example 2) that, compared to the control group (normal mouse cells; Cbfβ^{fl/fl}), the expression of Runx1, Runx2, VEGF, PDGF-BB, PDGFRB, RANKL, and RANKL/OPG was significantly increased in the Cbfβ-deleted mouse cell (Cbfβ^{Δvc/Δvc}), and the expression of Cbfβ, OPG, PPARG, OPN, and ANGPTL4 was significantly decreased. In addition, as shown in FIG. 8B, it was found through ELISA (Experimental Example 11) that RANKL expression significantly increased in the Cbfβ-deleted mouse cells (HE CM and HO CM) compared to the control group (WT CM). From the results, it was determined that Cbfβ suppressed osteoclast differentiation through regulation of the expression of genes associated with osteoclast differentiation.

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the attached claims and their equivalents.

1. A method for preventing or treating a geriatric disease, comprising administering a pharmaceutical composition comprising a pharmaceutically effective amount of a core-binding factor subunit beta (Cbfβ)-expressing vascular smooth muscle cell or a culture fluid thereof as an active ingredient to a subject in need of preventing or treating the geriatric disease.

2. The method of claim 1, wherein the Cbfβ-expressing vascular smooth muscle cell or culture fluid thereof suppresses blood pressure increase; or osteoclast differentiation.

3. The method of claim 1, wherein the geriatric disease is a vascular aging disease or a bone disease.

4. The method of claim 3, wherein the vascular aging disease is one or more selected from the group consisting of vascular calcification, osteoporosis, osteoarthritis, arteriosclerosis, fatty liver, liver fibrosis, diabetes, and dementia.

5. The method of claim 3, wherein the bone disease is one or more selected from the group consisting of fracture, osteoarthritis, rheumatoid arthritis, osteoporosis, and osteomalacia.

6. A method for preventing or ameliorating a geriatric disease, comprising administering a health functional composition comprising a therapeutically effective amount of a core-binding factor subunit beta (Cbfβ)-expressing vascular smooth muscle cell or a culture fluid thereof as an active ingredient to a subject in need of preventing or treating the geriatric disease.

7. A method for preventing or treating a geriatric disease, comprising administering a pharmaceutically effective amount of a pharmaceutical composition comprising an agent which induces expression or activity of a core-binding factor subunit beta (Cbfβ) protein or a gene encoding the same as an active ingredient to a subject in need of preventing or treating the geriatric disease.

8-18. (canceled)
